(19) United States
(12) Patent Application Publication
(10) Pub. No.: US 2014/0194925 A1
(43) Pub. Date: Jul. 10, 2014

(54) METHOD AND SYSTEM FOR CLOSING A VASCULAR HOLE

(71) Applicant: ST. JUDE MEDICAL PUERTO RICO LLC, Caguas, PR (US)

(72) Inventors: JyueBoon Lim, New Brighton, MN (US); Catherine A. Pipenhagen, Plymouth, MN (US); Brian J. Edquist, San Francisco, CA (US); Thomas A. Savard, Arden Hills, MN (US); Rachael A. Crabb, Helsingor (DK)

(73) Assignee: ST. JUDE MEDICAL PUERTO RICO LLC

(21) Appl. No.: 14/116,309

(22) PCT Filed: May 16, 2012
(86) PCT No.: PCT/US2012/038030

§ 371 Date: Jan. 6, 2014

Related U.S. Application Data
(60) Provisional application No. 61/487,474, filed on May 18, 2011.

(51) Int. Cl. A61B 17/00

(52) U.S. Cl. CPC A61B 17/0057
USPC 606/213

(57) ABSTRACT

Various embodiments of methods for closing vascular holes and associated vascular closure devices are described herein. The methods, generally speaking, use hemostatic devices intended to stop bleeding by closing vascular access puncture sites following percutaneous diagnostic or therapeutic procedures. The methods may include positioning a sealing plug in the vascular hole. In one embodiment, the sealing plug may extend through the vascular hole into the vessel. The sealing plug may expand and thereby create a lip inside the vessel that holds the sealing plug in place.

### RELATED APPLICATION

This claims the benefit or U.S. Provisional Application No. 61/487,474, filed 18 May 2011, which is hereby incorporated by reference herein in its entirety.

### BACKGROUND

Catheter based diagnostic and interventional procedures such as angiograms, balloon angioplasty, stenting, atherectomy, thrombectomy, device placement, etc., are commonly employed to treat patients with vascular obstructions or other abnormalities accessible through the vasculature of the human body. Such interventions are less traumatic to the body than previous surgical interventions and therefore are growing in use.

To gain access to the vasculature, the Seldinger technique is commonly employed. This involves placing a small gauge hollow needle through the skin to intersect the desired vessel, commonly, but not always, the femoral artery in the groin area. The needle is known to have punctured the vessel wall when blood exits the needle at the proximal end. A guidewire is inserted through the needle into the vessel and the needle is removed.

A dilator with a lumen sized to fit the guidewire has a leading tapered end and an outside diameter sized to fit closely in an introducer sheath placed over it. The size of the introducer sheath is selected (typically 5-8 Fr) to accommodate the catheters anticipated to be used in the procedure. The introducer sheath and tapered dilator are advanced together over the guidewire through the skin and into the vessel. The dilator and guidewire are then removed, since the vascular pathway from outside the body through the sheath and into the vessel has been established. A self-sealing stretchable valve at the proximal end of the introducer sheath minimizes blood loss from the introducer sheath during the procedure.

Following the procedure and after all of the catheters and guidewires have been removed from the body, the introducer sheath is removed from the artery. Historically, this has been done by exerting manual pressure on the vessel upstream from the access site to lower blood pressure while the introducer sheath was removed. Once removed, manual pressure is applied directly to the skin above the access puncture for about thirty minutes to inhibit blood loss until the body's natural clotting process sealed the puncture. This technique is generally considered unsatisfactory because it is uncomfortable for the patient and requires a significant amount of nursing staff time.

Sealing the artery by manual compression is rapidly being replaced by medical devices designed to provide a vascular puncture seal in less time. These devices are intended to be effective and easy to use by medical personnel. The devices range from mechanical suturing devices to collagen plugs, vascular clips, staples, and use of adhesives and sealants. These different approaches have had varying degrees of success and ease of use.

One of the more commonly used devices for closing vessel punctures achieves hemostasis at the vessel puncture site by closing the puncture with an absorbable intra-vessel (e.g., intra-arterial) anchor and an extra-vessel (e.g., extra-arterial) collagen sponge. The anchor and collagen are held together with a self tightening suture loop and slip knot, which, when tightened, sandwiches the puncture hole between the anchor and the collagen sponge. The device is easy to use and the bio-absorbable anchor, collagen, and suture sandwich seals the vessel quickly, is more comfortable for the patient, saves valuable nurse time, and allows early patient ambulation.

Although such collagen devices may be highly effective, a substantial number of punctures in, for example, the femoral artery, may result in greater difficulty or greater chances of complications in treating the patient with such a device. Factors that may limit use of this device include presence of severe peripheral vascular disease, poor needle stick location (too high or too low), or small vessel size which interferes with anchor placement and prevents proper seating of the anchor against the arterial wall.

In an effort to overcome some of these problems, vascular closure devices have been developed that deposit a plug outside the vessel with no component inside the vessel. Such devices may generally require, however, consistently placing the plug near the arterial wall. Unfortunately, these devices may suffer from a number of drawbacks. For example, the pressure exerted on the plug as the heart beats may cause the plug to move away from the hole in the vessel resulting in a hemotoma, late bleeding, or other complications. The plug may not seal the puncture tract/hole in the blood vessel sufficiently to prevent leakage.

Accordingly, it would be desirable to provide an improved vascular closure device or vascular sealing device that addresses these and other shortcomings in the art. A number of embodiments of such improved vascular closure devices are shown and described herein.

### SUMMARY

Various embodiments of methods for closing vascular holes and associated vascular closure devices are described herein. The methods, generally speaking, use hemostatic devices intended to stop bleeding by sealing vessels that have been accessed surgically, such as vascular access puncture sites following percutaneous diagnostic or therapeutic procedures. It should be appreciated that the methods and devices described herein may be used to close any puncture in any bodily lumen although they are most commonly used to close arteriotomies. It should also be appreciated that the methods and devices are not limited to use on humans. They may be used to close vascular punctures in suitable animal species as well.

In one embodiment, a method of closing a vascular hole comprises inserting a portion of a vascular closure device into a tissue tract. The vascular closure device includes a sealing plug and a delivery system. The method includes deploying the sealing plug with the sealing system so that at least a portion of the sealing plug is positioned in the vascular hole. In some arrangements, the sealing plug is arranged with no portion thereof extending into a lumen defined by the vessel.

In another embodiment, a method of closing a vascular hole comprises inserting a portion of a vascular closure device into a tissue tract. The vascular closure device includes a sealing plug and a delivery system. The method includes deploying the sealing plug with the delivery system so that a distal portion of the sealing plug extends through the vascular hole and into a lumen defined by the vessel.

In another embodiment, a vascular closure device comprises a delivery system (e.g., a carrier tube) and a sealing plug carried by the delivery system. The sealing plug is configured to be deployed to close a vascular hole. The delivery system is configured to deploy the sealing plug so that a portion of the sealing plug is positioned in the vascular hole. In some arrangements, at least portions of the sealing plug also extend through the vascular hole.

The term “nonthrombogenic” and other like terms (e.g., not thrombogenic and nonthrombogenic etc.) are used to indicate that the material thus described does not cause significant clotting when used in the manner described herein. Thus, a nonthrombogenic material may be thrombogenic to some degree so long as the material is still suitable to be used in the manner described herein

The foregoing and other features, utilities, and advantages of the subject matter described herein will be apparent from the following more particular description of certain embodiments as illustrated in the accompanying drawings.

### DRAWINGS

FIG. 1 is a schematic cross-sectional view showing a sealing plug deployed through a vascular hole.

FIG. 2 is a schematic cross-sectional view showing the sealing plug after it has been exposed to bodily fluids that cause the sealing plug to expand.

FIG. 3 is a schematic cross-sectional view showing the flow of fluid in the vessel around the sealing plug shown in FIG. 2.

### DETAILED DESCRIPTION

A number of embodiments of methods for closing vascular holes and associated vascular closure devices are described herein. The methods, generally speaking, use hemostatic devices intended to stop bleeding by closing vascular access puncture sites following percutaneous diagnostic or therapeutic procedures. It should be appreciated that the methods and devices described herein may be used to close any puncture in any bodily lumen although they are most commonly used to close arteriotomies in the femoral artery.

A method for closing a vascular hole may include deploying a sealing plug 100 in or through the vascular hole 102 in a bodily vessel 104. In one embodiment, the sealing plug is deployed in or through the vascular hole 102 without the use of a separate anchor component positioned in the vessel. Eliminating the anchor component allows the method and associated devices to be used to close vascular holes that may otherwise be ineligible to be closed with a device.

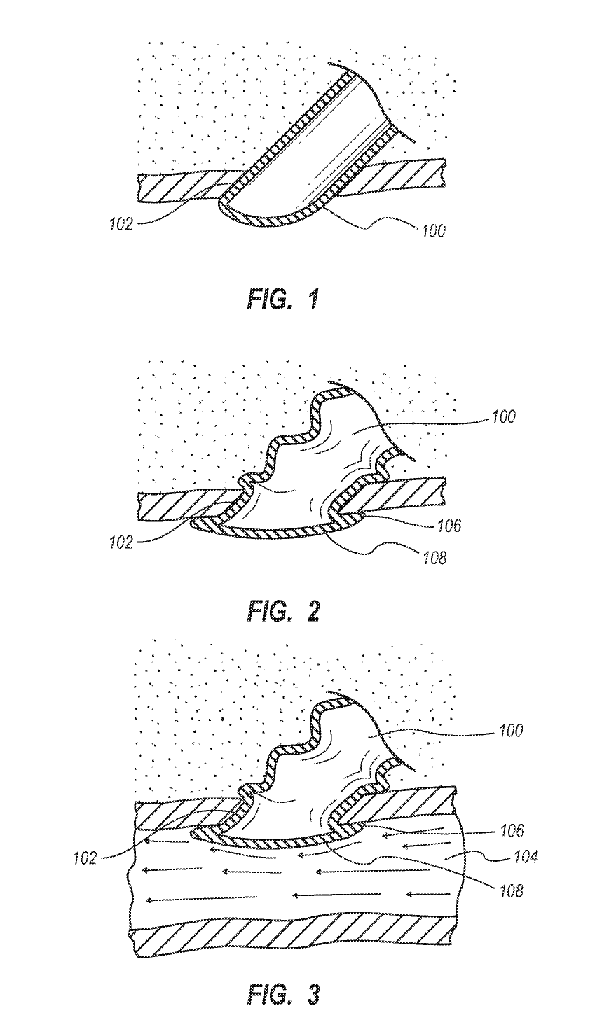

The sealing plug 100 is inserted into the hole 102 in the vessel 104 and allowed to expand. This is shown in FIGS. 1-3. The sealing plug 100 is shown initially positioned in the hole 102 in FIG. 1 and arranged at an angle of about 45 degrees relative to a length direction of the vessel or a longitudinal direction of fluid flow in the vessel (see FIG. 3). The angled arrangement, size and shape of the sealing plug 100 and hole 102 relative to the vessel 104 as shown in FIGS. 1-3 is schematic and not to scale. Other shapes, sizes and angled arrangements of the sealing plug 100 and hole 102 relative to the vessel 104 are possible. For example, at least one of the sealing plug 100 and hole 102 may be arranged at any non-parallel angle relative to a length dimension of the vessel, such as at an angle in the range of about 30 degrees to about 80 degrees.

With at least a portion of the sealing plug 100 positioned in the hole 102, the sealing plug is expanded as shown in FIG. 2. The sealing plug 100 expands to the peripheral edges or wall that defines the hole 102. In some arrangements, the sealing plug 100 exerts a force against the peripheral edges or wall that defines the hole 102. The expanded sealing plug 100 is held in place, thereby preventing or reducing hematomas. In some arrangements, the sealing plug 100 extends at least partially into the hole 102, but does not extend into the vessel interior.

The sealing plug may expand as a result of contact with bodily fluids. In one example, the sealing plug absorbs bodily fluid into pores such as in a sponge, thereby causing the sealing plug to expand. In other examples, the sealing plug may include a hydrophilic material, a chemically reactive material, or a temperature-activated material that result in expansion of the sealing plug when exposed to bodily fluids. Alternatively, the sealing plug may include a mechanical expansion feature, whereby activation of the mechanical feature causes the sealing plug to expand within the hole 102. The sealing plug may also comprise adhesive properties that promote engagement of the sealing plug to the tissue tract or the peripheral edges or wall that defines the hole 102. In a yet further embodiment, a portion of the sealing plug not exposed to bodily fluid within the vessel may be blood activated to promote the natural clotting mechanisms in the area adjacent the hole 102. Such clotting, alone or in combination with other features of the sealing plug such as the adhesive feature or the mechanical expansion forces described above may help maintain the sealing plug position relative to the hole 102.

In one embodiment, the sealing plug 100 may extend through the hole 102 in the vessel 104. This is also depicted in FIGS. 1-3. As the sealing plug 100 expands, the sealing plug 100 forms a lip or rim 106 that extends radially outward relative to a central axis of the hole 102 beyond the peripheral edges of the hole 102. The lip 106 acts to anchor or hold the sealing plug 100 in position. Moreover, the lip 106 gives the sealing plug 100 a unique robustness under motion that is not achievable with other closure devices. Typically, the sealing plug 100 does not extend so far into the vessel 104 that it substantially blocks the flow of fluid flowing through the vessel 104.

The distal tip 108 of the sealing plug 100 may be rounded or have other shapes and sizes that help minimize turbulent fluid flow through the vessel 104. FIG. 3 shows the fluid flow in the vessel 104 with the sealing plug 100 in position. In some arrangements, the fluid flow may be largely laminar and more orderly than would otherwise be the case if the distal tip 108 was not rounded. In one example, the sealing plug 100 may have a generally cylindrical shape (also referred to as a “bullet” shape) with a circular cross section along its length. Other constructions and cross sectional shapes for the sealing plug 100 are possible. A rounded tip at the end of a cylindrical shaped sealing plug 100 with a circular cross section may result in a domed or hemispherical shape tip that is exposed in the vessel 104.

The sealing plug 100 may include any one or a combination of different sealing materials. The sealing material may be any suitable material that is capable of effectively closing the hole in the vessel. In one embodiment, the sealing material expands in the presence of bodily fluids such as blood. For example, the sealing plug 100 may include a sealing material that expands quickly upon exposure to bodily fluids resulting in accelerated hemostasis. In some cases, accelerating hemostasis may reduce or prevent hematomas. In another embodiment, the sealing material may be nonthrombogenic. In yet another embodiment, the sealing material may be biodegradable. In other embodiments, the sealing material may include a hydrophilic material, a material that creates a chemical reaction in the presence of blood, a temperature activated material, or a material with adhesive properties. In yet another embodiment, the sealing material may be capable of being compressed and loaded into a delivery system that includes, for example, tubular member. The sealing material may also be capable of expanding to at least its original shape upon being ejected from the delivery system. The sealing material may have all or any suitable combination of these and other properties.

In one embodiment, at least a portion of the sealing plug 100 may be coated with a nonthrombogenic agent or an antithrombotic agent. The nonthrombogenic agent may be used with sealing material that is otherwise thrombogenic to make it suitable to be placed in or through the vascular hole. The nonthrombogenic agent may also be coated on nonthrombogenic sealing materials for an extra layer of defense against thrombosis. An example nonthrombogenic agent is polyglycolic acid. An example antithrombotic agent is Heparin.

Although the sealing plug 100 may include any suitable material, some examples of suitable sealing materials include collagen, gelatin, polyethyleneglycol, polyglycolic acid, polylactic acid, alginate, polyvinyl alcohol, hyaluronic acid, polyacrylic acid, chitosan, or any combination thereof.

Any of a number of suitable vascular closure devices and delivery systems may be used to deploy the sealing plug 100. For example, the techniques and devices shown in any of the following patent applications may be used to locate the hole 102 and deploy the sealing plug 100: U.S. patent application Ser. Nos. 11/968,020, 11/967,979, 11/967,896, 11/842,509, 11/605,603, 11/532,819, 11/419,941, 11/325,206, 11/197,382, 11/131,120, 11/130,688, 11/103,730, 11/103,257, 11/103,196, and 11/051,892, all of which are incorporated herein by reference in their entireties. Determination of the arteriotomy location may be performed using mechanical devices, electrical devices, pressure differential devices, and visual indicators. After determining the location of the arteriotomy, various devices and methods may be employed to deploy the sealing plug. In one example, the sealing plug is positioned within a portion of a delivery system, such as within a carrier tube, the sealing plug is position with the delivery device, and then the delivery device is withdrawn, leaving the sealing plug in place. In another example, the delivery system is properly positioned relative to the arteriotomy, and then the sealing plug is advanced distally through the delivery system (e.g., through the interior of a carrier tube) to the arteriotomy.

### Illustrative Embodiments

Reference is made in the following to a number of illustrative embodiments of the subject matter described herein. The following embodiments illustrate only a few selected embodiments that may include the various features, characteristics, and advantages of the subject matter as presently described. Accordingly, the following embodiments should not be considered as being comprehensive of all of the possible embodiments. Also, features and characteristics of one embodiment can and should be interpreted to equally apply to other embodiments or be used in combination with any number of other features from the various embodiments to provide further additional embodiments, which may describe subject matter having a scope that varies (e.g., broader, etc.) from the particular embodiments explained below. Accordingly, any combination of any of the subject matter described herein is contemplated.

In one embodiment, a method of closing a vascular hole comprises: inserting a vascular closure device into a tissue tract, the vascular closure device including a sealing plug; and deploying the sealing plug so that the sealing plug is positioned in the vascular hole. The method may comprise withdrawal of the vascular closure device from the tissue tract. The method may comprise deploying the sealing plug so that a distal portion of the sealing plug extends through the vascular hole. The sealing plug may expand outward against peripheral edges defining the vascular hole to hold the sealing plug in position. The sealing plug may be nonthrombogenic. The sealing plug may be coated with a nonthrombogenic agent. The sealing plug may expand when exposed to bodily fluids. The sealing plug may be compressed in the vascular closure device. The sealing plug may be biodegradable.

In another embodiment, a method of closing a vascular hole comprises: inserting a vascular closure device into a tissue tract, the vascular closure device including a sealing plug; and deploying the sealing plug so that a distal portion of the sealing plug extends through the vascular hole. The distal portion of the sealing plug may extend beyond the vascular hole in a distal direction relative to the operator of the vascular closure device. The distal portion of the sealing plug may be rounded or have other shapes. The sealing plug may expand outward against peripheral edges defining the vascular hole to hold the sealing plug in position. The distal portion of the sealing plug may expand beyond the peripheral edges to create a lip that holds the sealing plug in position. The sealing plug may be nonthrombogenic. The sealing plug may be coated with a nonthrombogenic agent. The sealing plug may expand when exposed to bodily fluids. The sealing plug may be compressed in the vascular closure device. The sealing plug may be biodegradable.

In another embodiment, a vascular closure device comprises: a delivery system; and a sealing plug carried by the delivery system, the sealing plug being configured to be deployed to close a vascular hole; wherein the carrier system is configured to deploy the sealing plug so that the sealing plug is positioned in the vascular hole.

The terms recited in the claims should be given their ordinary and customary meaning as determined by reference to relevant entries (e.g., definition of “plane” as a carpenter's tool would not be relevant to the use of the term “plane” when used to refer to an airplane, etc.) in dictionaries (e.g., widely used general reference dictionaries and/or relevant technical dictionaries), commonly understood meanings by those in the art, etc., with the understanding that the broadest meaning imparted by any one or combination of these sources should be given to the claim terms (e.g., two or more relevant dictionary entries should be combined to provide the broadest meaning of the combination of entries, etc.) subject only to the following exceptions: (a) if a term is used herein in a manner more expansive than its ordinary and customary meaning, the term should be given its ordinary and customary meaning plus the additional expansive meaning, or (b) if a term has been explicitly defined to have a different meaning by reciting the term followed by the phrase “as used herein shall mean” or similar language (e.g., “herein this term means,” “as defined herein,” “for the purposes of this disclosure [the term] shall mean,” etc.). References to specific examples, use of “i.e.,” use of the word “invention,” etc., are not meant to invoke exception (b) or otherwise restrict the scope of the recited claim terms. Other than situations where exception (b) applies, nothing contained herein should be considered a disclaimer or disavowal of claim scope. The subject matter recited in the claims is not coextensive with and should not be interpreted to be coextensive with any particular embodiment, feature, or combination of features shown herein. This is true even if only a single embodiment of the particular feature or combination of features is illustrated and described herein. Thus, the appended claims should be read to be given their broadest interpretation in view of the prior art and the ordinary meaning of the claim terms.

As used herein, spatial or directional terms, such as “left,” “right,” “front,” “back,” and the like, relate to the subject matter as it is shown in the drawing FIGS. However, it is to be understood that the subject matter described herein may assume various alternative orientations and, accordingly, such terms are not to be considered as limiting. Furthermore, as used herein (i.e., in the claims and the specification), articles such as “the,” “a,” and “an” may connote the singular or plural. Also, as used herein, the word “or” when used without a preceding “either” (or other similar language indicating that “or” is unequivocally meant to be exclusive—e.g., only one of x or y, etc.) shall be interpreted to be inclusive (e.g., “x or y” means one or both x or y). Likewise, as used herein, the term “and/or” shall also be interpreted to be inclusive (e.g., “x and/or y” means one or both x or y). In situations where “and/or” or “or” are used as a conjunction for a group of three or more items, the group should be interpreted to include one item alone, all of the items together, or any combination or number of the items. Moreover, terms used in the specification and claims such as have, having, include, and including should be construed to be synonymous with the terms comprise and comprising.

Unless otherwise indicated, all numbers or expressions, such as those expressing dimensions, physical characteristics, etc. used in the specification (other than the claims) are understood as modified in all instances by the term “approximately.” At the very least, and not as an attempt to limit the application of the doctrine of equivalents to the claims, each numerical parameter recited in the specification or claims which is modified by the term “approximately” should at least be construed in light of the number of recited significant digits and by applying ordinary rounding techniques. Moreover, all ranges disclosed herein are to be understood to encompass and provide support for claims that recite any and all subranges or any and all individual values subsumed therein. For example, a stated range of 1 to 10 should be considered to include and provide support for claims that recite any and all subranges or individual values that are between and/or inclusive of the minimum value of 1 and the maximum value of 10; that is, all subranges beginning with a minimum value of 1 or more and ending with a maximum value of 10 or less (e.g., 5.5 to 10, 2.34 to 3.56, and so forth) or any values from 1 to 10 (e.g., 3, 5.8, 9.9994, and so forth).

What is claimed is:

1. A method of closing a vascular hole comprising:
inserting a portion of a vascular closure device into a tissue tract, the vascular closure device including a sealing plug and a delivery system; and
deploying the sealing plug with the delivery system so that at least a portion of the sealing plug is positioned in the vascular hole;
wherein the sealing plug expands to peripheral edges defining the vascular hole to hold the sealing plug in position.

2. The method of claim 1 comprising withdrawing the delivery system from the tissue tract with at least a portion of the sealing plug maintained in the vascular hole.

3. The method of claim 1 comprising deploying the sealing plug so that a distal portion of the sealing plug extends through the vascular hole.

4. The method of claim 1 wherein at least a portion of the sealing plug is nonthrombogenic.

5. The method of claim 1 wherein at least a portion of the sealing plug is coated with a nonthrombogenic agent or an antithrombotic agent.

6. The method of claim 1 wherein the sealing plug expands when exposed to bodily fluids.

7. The method of claim 1 wherein the sealing plug is compressed in the delivery system prior to deploying the sealing plug.

8. The method of claim 1 wherein the sealing plug is biodegradable.

9. A method of closing a vascular hole comprising:
inserting at least a portion of a vascular closure device into a tissue tract, the vascular closure device including a sealing plug and a delivery system; and
deploying the sealing plug with the delivery system so that a distal portion of the sealing plug extends through the vascular hole.

10. The method of claim 9 wherein a distal portion of the sealing plug is shaped to minimize turbulent fluid flow in a vessel that defines the vascular hole.

11. The method of claim 9 wherein the sealing plug expands to peripheral edges defining the vascular hole to hold the sealing plug in position.

12. The method of claim 14 wherein the distal portion of the sealing plug expands radially outward beyond the peripheral edges to create a lip that engages the tissue tract to hold the sealing plug in position.

13. The method of claim 9 wherein at least a portion of the sealing plug is nonthrombogenic or antithrombotic.

14. The method of claim 9 wherein at least a portion of the sealing plug is coated with a nonthrombogenic agent or an antithrombotic agent.

15. The method of claim 9 wherein the sealing plug expands when exposed to bodily fluids.

16. The method of claim 9 wherein the sealing plug is compressed in the delivery system prior to deploying the sealing member.

17. The method of claim 9 wherein the sealing plug is biodegradable.

18. The method of claim 15 wherein the sealing plug comprises a temperature activated material.

19. The method of claim 15, wherein the sealing plug comprises a material that absorbs bodily fluids.

20. A vascular closure device comprising:
a delivery system; and
a sealing plug carried by the delivery system, the sealing plug being configured to be deployed to close a vascular hole;
wherein the delivery system is configured to deploy the sealing plug so that at least a portion of the sealing plug is positioned in the vascular hole.

21. The vascular closure device of claim 20 wherein the delivery system includes a carrier tube.

22. The vascular closure device of claim 21, wherein the sealing plug is compressed within the carrier tube prior to being deployed.

23. The vascular closure device of claim 20, wherein at least a portion of the sealing plug extends through the vascular hole.
